(19) United States
(12) Patent Application Publication
(10) Pub. No.: US 2020/0178569 A1
(43) Pub. Date: Jun. 11, 2020

(54) Method of Coating an Edible Thermoplastic Pet Chew

(71) Applicants: Alvin Kershman, Chesterfield, MO (US); Jeff L. Shear, Bonita Springs, FL (US)

(72) Inventors: Alvin Kershman, Chesterfield, MO (US); Jeff L. Shear, Bonita Springs, FL (US)

(21) Appl. No.: 16/792,011

(22) Filed: Feb. 14, 2020

Related U.S. Application Data
(63) Continuation-in-part of application No. 15/494,196, filed on Apr. 21, 2017.
(60) Provisional application No. 62/329,376, filed on Apr. 29, 2016.

(51) Int. Cl. A23K 40/30; A23K 20/163; A23K 20/189; A23K 20/174; A23K 20/158; A23K 20/179; A23K 20/111; A23K 20/20; A23K 50/40; A61K 9/00; A61K 33/30; A61K 31/155

(52) U.S. Cl. CPC A23K 40/30; A23K 20/163; A23K 20/189; A23K 20/174; A23K 20/158; A61K 31/155; A23K 20/111; A23K 20/30; A23K 50/40; A61K 9/0056; A61K 33/30; A23K 20/179

(57) ABSTRACT

A method of preparing an edible thermoplastic nutritious pet chew having an edible film coating comprising applying an aqueous solution or suspension of a cellulose ester and at least one hydrolysable nutrient or at least one heat labile nutrient to the uncoated pet chew; and drying the pet chew at a low temperature to form an edible film coating containing the nutrient, wherein the coating adheres to the pet chew, and wherein the nutrient in the coating is not degraded by heat and hydrolysis.

### CROSS-REFERENCE TO RELATED APPLICATIONS

This application is a Continuation-in-Part of pending utility patent application Ser. No. 15/494,196 filed Apr. 21, 2017, which claims the benefit of provisional patent application No. 62/329,376 filed on Apr. 29, 2016, which are both hereby incorporated by reference.

### STATEMENT REGARDING FEDERALLY SPONSORED RESEARCH

Not Applicable.

### APPENDIX

Not Applicable.

### BACKGROUND OF THE INVENTION

### Field of the Invention

### Related Art

The present invention relates to method of coating an edible thermoplastic and nutritious pet chew with an edible polymer film and the pet chew so coated. The method uses an aqueous solution to apply the edible polymer film containing nutrients to the surface of the pet chew.

Edible thermoplastic and nutritious pet chew products are disclosed in U.S. Pat. Nos. 6,455,083 and 6,379,725, which are hereby incorporated by reference. Both of these products contain a combination of protein, water and edible fiber. The products are made by injection molding, compression molding, transfer molding, pressure forming or extrusion. To make these thermoplastic products by the above methods, it is necessary to heat the pre-formed product to a temperature in the range of from about 90 to 150 degrees C. These elevated temperatures destroy desirable heat labile nutrients, such as enzymes, Vitamin C, Vitamin B₁, Vitamin B₂, Vitamin D and carotenes. The present invention uses coatings to provide the desirable labile nutrients with the pet chew.

Food coatings used in the prior art are described in the references listed below, which are hereby incorporated by reference: US20020121225 discloses a coating composition of a primary film former of a low viscosity propylene glycol alginate (PGA), a 1% aqueous solution of which has a viscosity in the range of about 1 to 500 mPa·s at 25° C. The PGA preferably is used in combination a surface active agent, and optionally such additional ingredients as a filler, a coloring agent, or combination of these. US20080118627 discloses a liquid coating formulation comprising a polymer selected from the group consisting of alginate, carageenan, carboxymethylcellulose, and hydroxypropylmethylcellulose in an amount from about 1% to 20%, one or more surfactants in an amount in an amount of 0.1 to 10%, one or more flavor in a sufficient amount, and water to give 100%. None of the above references disclose the claimed thermoplastic chew toy coated with nutrients contained in a polymer.

### SUMMARY OF THE INVENTION

A method of preparing an edible thermoplastic nutritious pet chew having an edible film coating comprising applying an aqueous solution or suspension of a cellulose ester and at least one hydrolysable nutrient and/or at least one heat labile nutrient to the uncoated pet chew; drying the pet chew at a low temperature to form an edible film coating containing the nutrient, wherein the coating adheres to the pet chew, and wherein the nutrient is not degraded by heat and hydrolysis.

An edible thermoplastic nutritious pet chew having an edible film coating containing at least one hydrolysable nutrient or at least one heat labile nutrient, wherein the edible film coating containing the nutrient adheres to the pet chew, and wherein the nutrient is degraded by neither heat nor hydrolysis.

A method of preparing a dental pet chew containing a dental germicidal product comprising applying an aqueous solution of a cellulose ester containing a germicidal product to the pet chew and drying the pet chew to from an edible film coating that adheres to the pet chew.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE PREFERRED EMBODIMENTS

Film coating are used for coating (also called “enveloping”) a substrate such as solid pharmaceutical forms (e.g. tablets, capsules), foodstuffs (e.g. chewing gum, breakfast cereals), agricultural products (e.g. seed, fruits) and the like. Film coatings are applied in dissolved or dispersed form to such a substrate, and a coherent film remains on the substrate after evaporation of the solvent or dispersant. The objectives of the application of film coatings depends on the area of application. Film coatings on pharmaceutical forms are intended to provide temporary protection from the spatial surroundings and thus avoid adverse effects on the substrate through atmospheric oxygen, moisture, light, heat or gastric juice. It is possible thereby, for example, to ensure controlled delivery of active ingredient by employing, a coating which is resistant to gastric juice and soluble in the small intestine and which protects the active ingredient and the pharmaceutical form from inactivation. In food technology, film coatings likewise have a protective function (e.g. suppression of penetration of moisture or loss of flavor). Besides the protective function it is also possible to add flavorings or to mask an unpleasant or bitter taste.

There are numerous types of coatings that are used on food products, such as confectionery sugar pan coatings, lower melting waxes and coco butter substitutes, gel coatings and cellulosic films. Cocoa Butter Substitutes include coconut oil or palm oil; cocoa butter replacer such as soybean oil, rapeseed oil and cottonseed oil; and cocoa utter equivalent, such as shea butter, illipe, sal nut, palm, mango kernel fat, and palm oils.

The preferred coatings are cellulose ethers. More preferably, the coatings are aqueous coatings, which provide better adherence to the edible bone, but have the disadvantage of having a longer drying time at a lower temperature.

Edible film coatings can be made from a cellulose ether. Preferably, the cellulose ether is a water soluble cellulose ether. Nonlimiting examples of water soluble cellulose ethers include carboxy-C₁-C₃-alkyl celluloses, such as carboxymethyl celluloses; carboxy-C₁-C₃-alkyl hydroxy-C₁-C₃-alkyl celluloses, such as carboxymethyl hydroxyethyl celluloses; C₁-C₃-alkyl celluloses, such as methylcelluloses; C₁-C₃-alkyl hydroxy-C₁₋₃-alkyl celluloses, such as hydroxyethyl methylcelluloses, hydroxypropyl methylcelluloses (HPMC) or ethyl hydroxyethyl celluloses; hydroxy-C1-3-alkyl celluloses, such as hydroxyethyl celluloses or hydroxypropyl celluloses (HPC); mixed hydroxy-C₁-C₃-alkyl celluloses, such as hydroxyethyl hydroxypropyl celluloses, mixed C₁-C₃-alkyl celluloses, such as methyl ethyl celluloses, or alkoxy hydroxyethyl hydroxypropyl celluloses, the alkoxy group being straight-chain or branched and containing 2 to 8 carbon atoms. The most preferred coatings are from HPC and/or HPMC.

The preferred materials for use as alginate in the present invention are alginate salts such as sodium alginate. The alginate salt is converted to a heat irreversible gel structure by contact with a gelation agent or a setting solution which provides a source of alkaline earth metal ions, such as magnesium and preferably calcium ions. A wide variety of salts may be employed as the source of alkaline earth ions. Typical salts include calcium or magnesium chlorides, carbonates, lactates and phosphates. However, any other alkaline earth salts which will provide a source of the metal ion may be employed.

Coatings can be applied a variety of ways, including dipping and spraying. A preferred method of coating that gives an even coating on uneven surfaces is robing, where the object is passed through a curtain of coating falling down over the object. This method is often used in coating confectionaries with milk chocolate.

Another method of coating is using a fluidized bed. The fluidized bed is formed when a quantity of a solid particulate substance (the thermoformed nutritious product) is placed under appropriate conditions to cause a solid/fluid mixture to behave as a fluid. This is usually achieved by the introduction of pressurized fluid through the particulate medium. This results in the medium then having many properties and characteristics of normal fluids, such as the ability to free-flow under gravity, or to be pumped using fluid type technologies.

A coating may be used to add desirable heat labile and/or hydrolysable nutrients to the pet chews, such as enzymes, vitamin C, vitamin B₁, vitamin B₂, vitamin D and carotenes. The coating is added to the chews after the product has been thermoformed and cooled. It may be applied as a solution or suspension. The process of preparing the coating solution differs, depending on which solution used to prepare it. For preparing solutions of hydroxypropylcellulose (HPC), commercially available as KLUCEL™ sold by Ashland Company or hydroxypropylmethylcellulose (HPMC) sold as HYPROMELLOSE™ by Dow Chemical Company, making an aqueous solution is typically a two step process. The granulated HPC or HPMC is dispersed in water that is 40 degrees C. to 80 degrees C. After dispersion, the water is either cooled or cold water added to bring the mixture to room temperature, where the HPC or HPMC dissolves and forms a solution. Heat labile nutrients are added to the cooled aqueous solutions. For HPC and HPMC, the celluloses both readily dissolve in polar solvents, such alcohols, but the coatings made from these solutions do not adhere to the thermoformed nutrient bones.

In a preferred embodiment, the coating is an aqueous solution or suspension of a cellulose ester and a hydrolysable additive and/or a heat labile additive to the pet chew; drying the pet chew at a low temperature to form an edible film coating containing an additive that adheres to the pet chew, wherein the additive is degraded by neither heat nor hydrolysis. The preferred concentration of the cellulose ester in the solution or suspension is in the range of from about 5 to 20 wt. %. The preferred cellulose ester for this coating is hydroxypropyl cellulose which provides an effective coating that adheres, yet dries under mild heating conditions effectively so that the additive does not hydrolyze on moisture held by the coating on the pet chew. Examples of additives that are unstable and susceptible to hydrolysis are water soluble vitamins, specifically vitamin C, thiamin, vitamin B₁₂ and folate, and enzymes, such as lipase.

When labile nutrients are applied with the coating, it is important that low heat be applied. Low heat, as applied to the coating should be 48 degrees C. or less to remove (dry) the solvent, since that would also destroy the added heat labile nutrients. To facilitate rapid drying at a low temperature, an organic solvent, such as an alcohol would be useful. However, it was discovered that the coating made using an alcohol solvent did not adhere to the pet chew, but the coating easily peeled off or flake off as the pet consumed it. The nutritional value of the coating was diminished or lost as it fell to the floor. Initially, water was the less-preferred solvent, as it required a longer drying time at the low temperature. However, unexpectedly, it was found that when water that was essentially free of organic solvents was used as the solvent for the polymer, the coating adhered to the pet chew, and was consumed with the pet chew. It did not flake off the pet chew.

Palatants are ingredient systems that are specially designed to make pet foods, treats, and supplements taste better, ensuring that pets receive the vital nutrients they need. Palatants entice a pet to consume a food, treat, or supplement that, while nutritious, may be inconsistent with their native diet. Palatants can be meat or vegetable based, and may be designed to meet a variety of claims (grain-free, limited ingredient, non-GMO, natural, low fat, etc.). Palatant components include proteins, yeasts, phosphates, antioxidants, antimicrobials, processing agents, and other ingredients. Palatant protein sources vary depending on desired performance targets, cost requirements, and brand claims. The protein can be vegetable or animal derived. Vegetable derived proteins come from many sources, including corn, soy, potato, and specialty grains. The desired vegetable protein source often depends on customer-specific needs. Common animal derived proteins are poultry, pork, and fish. A protein source that is available in one region—say kangaroo in Australia—may be considered uncommon in other regions of the world. The following description of the preferred embodiment(s) is merely exemplary in nature and is in no way intended to limit the invention, its application, or uses.

Palatants are often added to pet chews to make them more attractive to pets. However, they are an expensive ingredient, and when palatants are added to the full pet product, it significantly increases the cost of making the product. It was found that palatants could be added to the film coating, thereby flavoring the film coating and the pet product with a much smaller quantity per product while at the same time making the pet product attractive to the pet.

Lipids, such as coconut oil, can be added to the film coating and applied to the pet chew by one of two methods. An emulsion of the lipid can be prepared utilizing high HLB surfactants, and the emulsion added to the coating solution. High HLB surfactants include polysorbate 80 or polyoxysorbitan monolaurate (commercially available as the TWEEN® series of surfactants), polyglycerol-6 laurate, decaglycerol lauric acid esters, decaglycerol oleic acid esters and sucrose esters. Alternatively, the lipid can be dissolved in a polar solvent, and the solvent solution added to the aqueous coating solution.

Additives such as nutrients, palatants or lipids, are applied in the coating solution to the pet chew. The additives can be in solution, suspension or emulsion form. Once dried, all these additives are trapped in the film coating without damage or loss of activity.

In a preferred embodiment, a tooth cleaning film can be applied to the chew that, when the pet consumes the chew, the tooth cleaning additives coat the teeth. An example of a dental additive is chlorhexidine gluconate. Chlorhexidine gluconate is a germicidal product that reduces bacteria in the mouth. Zinc chloride can be added to enhance the effectiveness of the chlorihexidine gluconate.

Control Examples

Control 1: An 81% solution of sodium alginate was prepared as follows:

I. Sodium Alginate Solution (wt. g)

| | |
|---|---|
| Sodium Alginate | 81.00 |
| Glycerin | 4.00 |
| Deionized Water | 15.00 |
| | 100.00 |

1. Mix glycerin and water together
2. Slowly add sodium alginate using a lightning mixer, mixing until solubilized
3. Upon completion, the edible bones are ready to be dipped

II. Calcium Lactate Solution (wt. g)

| | |
|---|---|
| Calcium Lactate | 3.00 |
| Deionized Water | 97.00 |
| | 100.00 |

1. Dissolve calcium lactate in water for clear solution.

### Procedure

1. Put the edible bone in alginate solution. Drain off as much as possible. (usually takes about 15 seconds)
2. Drop into calcium lactate solution. Let sit 1 minute
3. Remove and let dry—air dry or low heat dryer oven.

A gel film coating was formed that adhered to the bone. This coated bone, while having a quality coating on the bone, retained too much moisture that would slowly degrade vitamins and other additives and was unacceptable.

Control 2:

A hard lipid coating was formed on the bone using a special lipid known as a “hard butter”, which has an SFI curve (solid fat index) equivalent to cocoa butter. However, when the bone was chewed by the dog, the coating flaked off, which was not acceptable to the pet owners/consumer, despite the fact that this makes a beautiful coating at acceptable processing temperatures.

Control 3:

A sugar coating containing gum Arabic followed by many coatings of sugar syrup was used to coat the edible bone. The sugar solution was prepared with vigorous stirring that replaced the high temperatures that are typically used to prepare sugar syrups. A pan coating method was used that required multiple coatings to adequately coat the edible bone. The process proved too cumbersome to be an acceptable choice because of the bone shape, and the coating must be put on in micro-layers to create a “jaw breaker” type of surface.

Control 4:

A coating of HPMC was prepared. While a coating could be made, the coating was not uniform, nor did it completely coat the edible bone. Since an aqueous mixture was used, the extended low-temperature drying time resulted in the hydrolyzation of various actives carried by the coating.

Control 5:

A coating of carboxymethyl cellulose was made but the coating was not uniform, nor did it completely coat the edible bone. The coating dissolved too slowly in the dog's mouth, which was very unattractive to the pet owner.

Control 6:

A coating of Klucel prepared using an organic solvent, which was desirable because of rapid drying. However, it was found that the coating, when the bone was chewed by an animal, flaked off and was not consumed.

### EXAMPLES OF THE INVENTION

The following examples are for illustration purposes only are not intended to limit the scope of this invention in any way.

### Example 1

An 18% solution of Klucel LF RM 822 (hydroxypropylcellulose) in deionized water was prepared. Edible bone chews were dipped in the solution and dried at 44 degrees C. A film coating was formed that adhered to the bone. Additional aqueous solutions of Klucel, concentrations (15%, 12% and 9%), were made and successfully coated onto a thermoformed bone.

### Example 2

A palatant-containing coating was made of a 9% solution of Klucel LF RM 822 in deionized water. Palatant 20012 RM1124 (1%) was added to the solution. Edible bone chews were dipped in the solution and dried at 44 degrees C. A film coating was formed that adhered to the bone.

### Example 3

A dental coating was made of 9.3 g of 20% chlorhexidine gluconate solution in water with 90.7 g of a solution of 15% Klucel in water. Edible bone chews were dipped in the solution and dried at 44 degrees C. A film coating was formed that adhered to the bone.

### Example 4

A dental coating with an emulsion was made of made of 9.1 g of 20% chlorhexidine gluconate solution in water, 72.9 g of a solution of 15% Klucel in water, and 18.0 g of a 50/50 coconut oil and surfactant emulsion. Edible bone chews were dipped in the solution and dried at 44 degrees C. A film coating was formed that adhered to the bone.

### Example 5

A dental coating with an emulsion was made of made of 9.0 g of 20% chlorhexidine gluconate solution in water, 72.5 g of a solution of 15% Klucel in water, 18.0 g of a 50/50 coconut oil and surfactant emulsion, and 0.5 g of a 5% aqueous solution of zinc chloride. Edible bone chews were dipped in the solution and dried at 44 degrees C. A film coating was formed that adhered to the bone.

The Examples 1-5 were prepared using Klucel (hydroxypropylcellulose). The edible bones prepared in Examples 1-5 were both effectively coated and dried sufficiently that nutrients that were unstable in the presence of moisture and tended to hydrolyze, did not hydrolyze. Further, the coating and drying process were all completed under low heat, so the heat labile additives were not destroyed in the process.

The embodiments were chosen and described to best explain the principles of the invention and its practical application to persons who are skilled in the art. As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A method of preparing a coated edible thermoplastic nutritious pet chew comprising:
applying an aqueous solution or suspension of a cellulose ether and one or more hydrolysable nutrient or one or more heat labile nutrient to an uncoated pet chew;
drying the coated pet chew at a low temperature to form an edible film coating containing an additive on the pet chew;
wherein the edible film coating adheres to the pet chew, and
wherein the hydrolysable nutrient and the heat labile nutrient is not degraded by heat and hydrolysis.

2. The method of claim 2, wherein the cellulose ether comprises hydroxypropyl cellulose.

3. The method of claim 2, wherein the drying temperature is 48 degrees C. or less.

4. The method of claim 3, wherein the a hydrolysable nutrient and/or a heat labile nutrient is selected from the group consisting of enzymes, vitamin C, vitamin B₁, vitamin B₂, vitamin D, carotenes, thiamin, vitamin Bit and folate.

5. The method of claim 1, wherein the aqueous suspension or solution further comprises a palatant.

6. The method of claim 1, wherein the aqueous suspension or solution further comprises a lipid and a surfactant.

7. An edible thermoplastic nutritious pet chew having an edible film coating made by the process comprising:
coating an uncoated thermoplastic edible pet chew with an aqueous suspension or solution comprising hydroxypropyl cellulose, water and at least one hydrolysable nutrient and/or at least one heat labile nutrient, and
drying the aqueous suspension or solution at 48 degrees C. or less to form the edible film coating on the thermoplastic pet chew;
wherein the edible film coating adheres to the pet chew and does not flake off as it is consumed; and
wherein the hydrolysable or the heat labile nutrient in the edible film coating is not degraded by heat and hydrolysis as the aqueous suspension or solution is dried to form the edible film coating on the edible thermoplastic nutritious pet chew.

8. The edible thermoplastic nutritious pet chew of claim 7, wherein the a hydrolysable nutrient and/or a heat labile nutrient is selected from the group consisting of enzymes, vitamin C, vitamin B₁, vitamin B₂, vitamin D, carotenes, thiamin, vitamin B₁₂ and folate.

9. The edible thermoplastic nutritious pet chew of claim 8, wherein the aqueous suspension or solution further comprises a palatant.

10. The edible thermoplastic nutritious pet chew of claim 9, wherein the aqueous suspension or solution further comprises a lipid and a surfactant.

11. An edible thermoplastic nutritious pet chew having an edible film coating made by the process comprising:
coating an uncoated edible thermoplastic pet chew with an aqueous suspension or solution comprising hydroxypropyl cellulose, water, a dental germicidal product, and at least one hydrolysable nutrient and/or at least one heat labile nutrient; and
drying the aqueous suspension or solution at 48 degrees C. or less to form the edible film coating on the thermoplastic chew;
wherein the water for the aqueous suspension or solution is water essentially free of organic solvents
wherein the edible film coating adheres to the pet chew and does not flake off when consumed; and
wherein the hydrolysable and heat labile nutrient in the edible film coating is not degraded by heat and hydrolysis as the aqueous suspension or solution is dried to form the edible film coating on the edible thermoplastic nutritious pet chew.

12. The edible pet chew of claim 11, further comprising zinc chloride.

13. The edible pet chew of claim 12, wherein the dental germicidal product is chlorhexidine gluconate.

14. The edible pet chew of claim 13, further comprising a lipid and a surfactant.

15. The edible thermoplastic nutritious pet chew of claim 7, wherein the concentration of the hydroxypropyl cellulose is the aqueous solution or suspension is in the range of about 5 wt. % to 20 wt. %.

16. The edible thermoplastic nutritious pet chew of claim 15, wherein the water for the aqueous suspension or solution consists essentially of water without organic solvents.

17. The edible thermoplastic nutritious pet chew of claim 16, wherein the aqueous suspension or solution consists of hydroxypropyl cellulose, water and at least one hydrolysable nutrient and/or at least one heat labile nutrient.
